(19) United States
(12) Patent Application Publication
(10) Pub. No.: US 2004/0122099 A1
(43) Pub. Date: Jun. 24, 2004

(54) Process for preparing S-(2-aminoethyl)-2-methyl-L-cysteine

(76) Inventor: Peter Guillaume Marie Wuts, Mattawan, MI (US)

Correspondence Address: PHARMACIA & UPJOHN, 301 HENRIETTA ST, 0228-32-LAW, KALAMAZOO, MI, 49007, US

(21) Appl. No.: 10/697,824

(22) Filed: Oct. 29, 2003

Related U.S. Application Data
(60) Provisional application No. 60/422,975, filed on Nov. 1, 2002.

(51) Int. Cl. A61K 31/198; C07C 323/25

(52) U.S. Cl. 514/562; 562/557

(57) ABSTRACT

The instant invention provides a process for the preparation of S-(2-aminoethyl)-2-methyl-L-cysteine comprised of the steps of (i) esterification of 2-methyl-L-cysteine; (ii) alkylation of the cysteine ester of step (i) to provide an N-protected S-(2-aminoethyl)-2-methyl-L-cysteine ester; and (iii) hydrolysis of the intermediate of step (ii) to provide the title compound in a salt free state.

### CROSS REFERENCE TO RELATED APPLICATION

This application claims the benefit of the following provisional application: U.S. Ser. No. 60/422,975, filed Nov. 1, 2002, under 35 USC 119(e)(i), which is incorporated herein by reference in its entirety.

### BACKGROUND OF THE INVENTION

S-(2-aminoethyl)-2-methyl-L-cysteine is an intermediate in the synthesis of S-[2-(ethanimidoylamino)ethyl]-2-methyl-L-cysteine, a nitric oxide synthase inhibitor useful in the treatment of inflammation disorders. The processes described for the preparation of S-[2-(ethanimidoylamino)ethyl]-2-methyl-L-cysteine, alternatively named S-[2-amino-3-(2-aminoethylsulfanyl)]-2-methyl-propionic acid, (U.S. patent application Publication Nos. 2002/0111493 and 2002/0019563) use complex methodology, costly ion exchange purification, produce product in modest yields, and employ ill-manipulable amino acid intermediates. In other instances, the use of N-protected amino acid esters have been used to provide tractable intermediates. Hydrolysis of these intermediates has been achieved with barium hydroxide in water and subsequent precipitation of barium as its carbonate salt (see, for example, Rojas-Rousseau, A., et al., Tetrahedron (2001), 57(16), 3389-3395; Labrecque, D., et al., Tetrahedron Letters (2001), 42(14), 2645-2648; Spielvogel, D., et al., Tetrahedron Letters (2000), 41(41), 7863; Bai, Y., et al., Journal of Carbohydrate Chemistry (2000), 19(7), 939-958; Spielvogel, D., et al., Tetrahedron Letters (2000), 41(41), 7863-7867) to give a solution of the free zwiterionic form of the amino acid. Similarly, calcium hydroxide has been described for the hydrolysis of carbamates (see, for example, Rank, A. W., et al., Can. J. Chem. (1981), 59(1), 27-33; Dornow, A., et al., Arch. Pharm. (1957), 290, 20-31; Bortnick, N. M., et al., J. Am. Chem. Soc. (1956), 78, 4358-61) and esters (see, for example, Hirth, G., et al., Helv. Chim. Acta (1985), 68(7), 1863-71). However, these methods have not been utilized in the preparation of the title compound and, thus, there exists a need for an improved process for the preparation of S-(2-aminoethyl)-2-methyl-L-cysteine.

### SUMMARY

The instant invention provides a process for the preparation of S-(2-aminoethyl)-2-methyl-L-cysteine comprised of the steps of (i) esterification of 2-methyl-L-cysteine; (ii) alkylation of the cysteine ester of step (i) to provide an N-protected S-(2-aminoethyl)-2-methyl-L-cysteine ester; and (iii) hydrolysis of the intermediate of step (ii) to provide the title compound in a salt free state.

### DETAILED DESCRIPTION

Preparation of S-[2-(ethanimidoylamino)ethyl]-2-methyl-L-cysteine, a nitric oxide synthase inhibitor, is best achieved with S-(2-aminoethyl)-2-methyl-L-cysteine in a salt free zwiterionic state. Thus, an object of this invention is to provide S-(2-aminoethyl)-2-methyl-L-cysteine in a salt free state with a cost effective process that utilizes easily handled and purified intermediates and environmentally safe reagents.

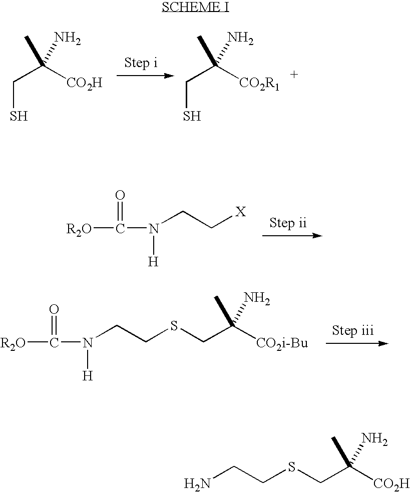

In step (i), 2-methyl-L-cysteine (Formula 1), or a suitable salt thereof,

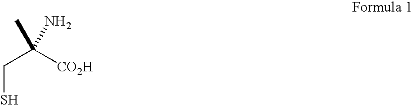

is reacted with an alcohol of Formula 2

R₁OH Formula 2

wherein R₁ is C-1 to C-8 alkyl or cycloalkyl

in the presence of a strong acid to give the cysteine ester of Formula 3, or salt thereof,

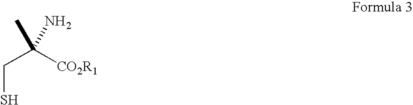

wherein R₁ is as defined for Formula 2.

Suitable strong acids are those with a pKₐ of less than 2 and include hydrochoric, hydrobromic, sulfuric, phosphoric, methanesulfonic, benzenesulfonic, toluenesulfonic and the like.

In Step 2, the thiolester of Formula 3, or a salt thereof, is reacted with an alkylating reagent of Formula 4;

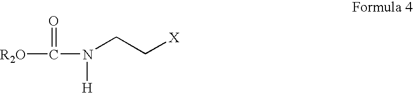

wherein R₂ is selected from the group Coo alkyl, C₁₋₆ alkenyl, trichloroethoxy, tri-C₁₋₆-alkylsilylethyl, benzyl and phenyl;

X is selected from the group —Cl, —Br, —I, —SO₂Ar, —SO₂CH₃, —SO₂CF₃;

in the presence of a suitable tertiary organic base to provide the cysteine derivative of Formula 5.

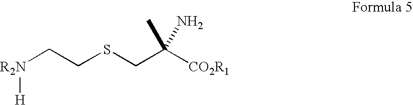

wherein R₁ and R₂ are as defined for Formulas 2 and 4.

Suitable tertiary organic bases are those with a pKₐ of greater than 9 and include such bases as triethylamine, tributylamine, diisopropylethylamine, pyridine, 4-N,N-dimethylpyridine, 1,8-diazabicyclo-[5.4.0]-undec-7-ene, 1,4-diazabicyclo-[2.2.2]-octane, 1,5-diazabicyclo-[4.3.0]-non-5-ene, tetramethylethylenediamine, and the like. Alternatively the reaction of 3 with 4 may be performed using a two phase mixture of a suitable immiscible organic solvent, an aqueous solvent and a phase transfer agent of the structure R₁R₂R₃R₄NX wherein R₁-R₄ are independently C₁ to C₁₆ alkyl or benzyl and X is a suitable counter ion such as Cl⁻, HSO₄⁻, Br⁻ I⁻ and the like.

The use of esters of Formula 5 provides intermediates that are easily isolated and purified by conventional means such as extraction with an organic solvent, distillation, chromatography and crystallization. They may also be purified by making suitable salts such as the benzoate, naproxenate, tartrate, maleate, malonate, succinate, suberate, fumarate, mandelate, phthalate, benzenesulfonate, p-tolylsulfonate, citrate, tartarate, methanesulfonate, and the like and purifying the salt.

In step iii, hydrolysis of the N-protected aminoesters of Formula 5 is readily achieved using calcium hydroxide as a base catalyst. The use of calcium hydroxide as the hydrolysis catalyst offers the advantage of being able to isolate the resultant amino acid in its zwiterionic form in a pure state without costly purification by ion exchange chromatography because the Ca⁺⁺ can be precipitated with carbon dioxide. A further advantage is that the procedure obviates environmentally hazardous reagents such as barium hydroxide more commonly used for this hydrolysis procedure

### EXAMPLES

The invention is further described in the following non-limiting examples.

### Example 1

### Preparation of L-Cysteine Isobutyl Ester.

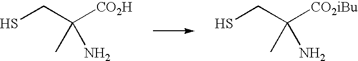

Acetyl chloride (3.11 ml) was cautiously added to isobutyl alcohol (25 ml) under a nitrogen atmosphere. 2-methyl cysteine hydrochloride (5.0 g, U.S. patent application Publication Nos. 2002/0111493 and 2002/0019563) was added and the mixture was refluxed for 14 hours. The isobutyl alcohol was removed under vacuum. Residual hydrochloric acid was removed by vacuum distillation with a second portion of isobutyl alcohol (50 ml) to leave the title ester as a viscous greenish-yellow oil.

### Example 2

### Preparation of N-Boc S-(2-aminoethyl)-2-methyl-L-cysteine, Isobutyl Ester; Use of Tertiary Organic Base.

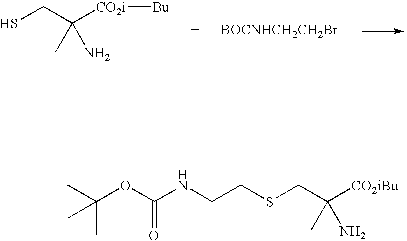

The ester of Example 1 was dissolved in isobutyl alcohol (50 ml) and the solution cooled to 0-5° C. Oxygen was displaced by purging the mixture with nitrogen. N-tert-butoxycarbonyl-2-bromoethylamine (13.06 g, available from Fluka, Milwaukee, Wis.) was added followed by diazabicycloundecane (13.1 ml) over 10 minutes. The mixture was allowed to warm to room temperature and stir for 14 hours. Methyl-t-butyl ether (MTBE, 100 ml) and water (50 ml) were added and the phases separated. The organic phase was washed with water (25 ml). The water wash was extracted with MTBE (25 ml) and the organic phases combined, dried over magnesium sulfate, and concentrated to give the title compound as an oil.

¹³C NMR (100 MHz, CDCl₃) δ 176.03, 155.72, 79.26, 71.41, 58.80, 42.58, 40.01, 33.97, 28.32, 27.67, 26.24, 19.01, 18.99

### Example 3

### Preparation of N-Boc S-(2-aminoethyl)-2-methyl-L-cysteine, Isobutyl Ester; Use of Phase Transfer Catalysis.

The isobutyl ester of Example 1 prepared from 2-methyl cysteine (10.0 g) was dissolved in isobutyl acetate and the solution cooled to 0-5° C. Tetrabutylammonium sulfate (0.985 g) was added followed by a solution of N-tert-butoxycarbonyl-2-bromoethylamine (12.3 g) in isobutyl acetate (50 ml). The mixture was flushed with nitrogen and a solution of sodium hydroxide (11.6 g) in water (50 ml) was slowly added while maintaining a reaction temperature of 0-5° C. After the addition of the sodium hydroxide solution, the mixture was allowed to warm to room temperature and stirred for one hour. The phases were separated and the aqueous phase extracted with isobutyl acetate (3×25 ml). The combined extracts were dried over magnesium sulfate and benzoic acid (7.1 g) added. Hexane was added and the mixture cooled and the precipitate collected and dried to give the benzoate salt of the title compound.

### Example 4

### Hydrolysis of N-Boc S-(2-aminoethyl)-2-methyl-L-cysteine, Isobutyl Ester; S-(2-aminoethyl)-2-methyl-L-cysteine.

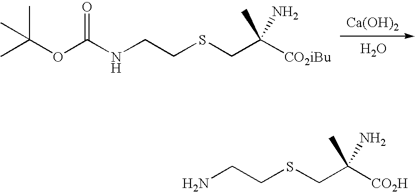

A mixture of N-Boc S-(2-aminoethyl)-2-methyl-L-cysteine, isobutyl ester (21.88 g), calcium hydroxide (14.53 g) and water (155 ml) was heated at 100° C. for fourteen hours. The mixture was cooled to room temperature and solid carbon dioxide added over 30 minutes to precipitate calcium as calcium carbonate. The mixture was filtered and concentrated to a volume of 20 ml. Isobutyl alcohol (120 ml) was added and the mixture distilled until no further water was seen in the azeotrope. The mixture is slowly cooled to room temperature and the resultant precipitate collected and dried to give S-(2-aminoethyl)-2-methyl-L-cysteine (8.76 g, 75%) as a white solid.

³C NMR (D₂O) δ 180.99, 59.87, 41.28, 39.26, 31.64, 25.03;

The described process then meets the objectives of producing S-(2-aminoethyl)-2-methyl-L-cysteine in high overall yield, with cost effectiveness, greater convenience and with environmentally sound reagents.

1. A process for preparing S-(2-aminoethyl)-2-methyl-L-cysteine comprising the steps of
(i) reacting an alcohol of Formula 2
R₁OH Formula 2
wherein R₁ is C-1 to C-8 alkyl or cycloalkyl with 2-methyl-L-cysteine (Formula 1), 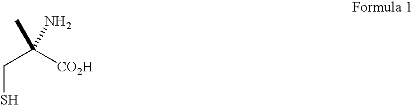
or a salt therof, to give the cysteine ester of Formula 3 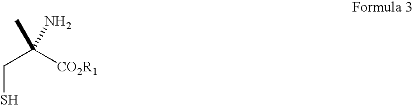
wherein R₁ is as defined for Formula 2;
(ii) reacting the thiolester of Formula 3 is with an alkylating reagent of Formula 4 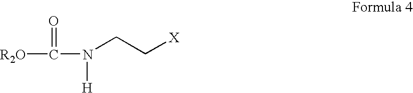
wherein R₂ is selected from the group C₁₋₆ alkyl, C₁₋₆ alkenyl, trichloroethoxy, tri-C₁₋₆-alkylsilylethyl, benzyl and phenyl;
X is selected from the Cl, Br, I, —SO₂Ar, —SO₂CH₃, —SO₂CF₃;
to provide the cysteine derivative of Formula 5, or a salt thereof, 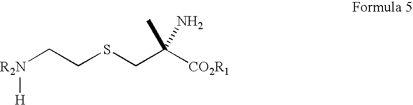
wherein R₁ and R₂ are as defined for Formulas 2 and 4; and
(iii) hydrolysis of the intermediate of step (ii) to provide the title compound.

2. A process according to claim 1 wherein R₁ is isobutyl.

3. A process according to claim 1 wherein R₂ is 1,1-dimethylethoxycarbonyl.

4. A process according to claim 1 wherein R₁ is C₁-C₈

5. A process according to claim 1 wherein R₂ is C₁₋₆ alkyl, C₁₋₆ alkenyl, trichloroethoxy, tri-C₁₋₆-alkylsilylethyl, benzyl or phenyl;

6. A process according to claim 1 further comprising the use of a tertiary organic base in an organic solvent in step ii.

7. A process according to claim 1 further comprising the use of a two phase mixture of a suitable immiscible organic solvent, an aqueous solvent and a phase transfer agent of the structure R₁R₂R₃R₄NX wherein R₁-R₄ are independently C₁ to C₁₆ alkyl or benzyl and X is a suitable counter ion.

8. A compound of Formula 1 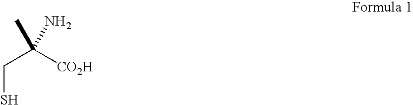
wherein R₁ is C-3 to C-8 alkyl or cycloalkyl.

9. A compound of Formula I wherein R₁ is isobutyl.

10. A compound of Formula II, or a pharmaceutically acceptable salt thereof, 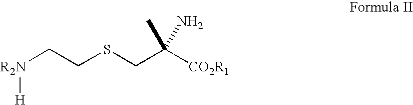
wherein wherein R₁ is C-3 to C-8 alkyl or cycloalkyl and R₂ is selected from the group C₁₋₆ alkyl, C₁₋₆ alkenyl, trichloroethoxy, tri-C₁₋₆-alkylsilylethyl, benzyl and phenyl.

11. A compound of claim 10 wherein the pharmaceutically acceptable salt is benzoate.

12. The use of a compound of Formula 1 for preparing compounds of Formula 2.

13. The use of a compound of Formula 2 for preparing S-(2-aminoethyl)-2-methyl-L-cysteine.
